(19) United States
(12) Patent Application Publication
(10) Pub. No.: US 2006/0222621 A1
(43) Pub. Date: Oct. 5, 2006

(54) Solid odor absorbers consisting of anodic oxide layers with active substance stored therein

(76) Inventors: Hubert Kuhn, Dusseldorfer (DE); Felix Muller, Velbert (DE); Jorg Peggau, Essen (DE); Gordon Thie, Hattingen (DE)

Correspondence Address: Leopold Presser, Scully, Scott, Murphy & Presser, 400 Garden City Plaza, Garden City, NY, 11530, US

(21) Appl. No.: 11/371,454

(22) Filed: Mar. 8, 2006

(30) Foreign Application Priority Data
Apr. 2, 2005 (DE) 10 2005 015 209.0

(51) Int. Cl. A61L 9/00

(52) U.S. Cl. 424/76.2; 977/900

(57) ABSTRACT

Solid odor absorbers including a solid carrier with a nanostructured surface and, incorporated therein, active zinc ricinoleate as an odor absorber are provided.

### DESCRIPTION

1. Field of the Invention

The present invention relates to solid odor absorbers including anodically prepared oxide layers as a carrier material with active substances stored therein for the irreversible binding of odorous and pollutant gases.

2. Background of the Invention

Odors can originate from diverse sources. Some are of natural origin and are produced by people and animals, others originate from agricultural or industrial production processes and can be released into the environment directly or via the corresponding products in order to be adsorbed into the articles of daily use, such as clothing, linen, upholstered furniture.

Cultural and esthetic standards have imposed a measure of the tolerable limits of such odors. The prior art therefore describes a large number of compositions and methods for suppressing or removing odors, in particular odors which are perceived as unpleasant.

These are essentially limited to their masking by other odors which are stronger, but generally perceived as pleasant, or their irreversible binding as a result of chemical reaction or physical adsorption and absorption to suitable materials.

The masking of odors is not a very effective method since it does not remove the odor formers and, therefore, can only be a short-term solution.

The irreversible binding of natural and artificial unpleasant odors is therefore the more effective option.

Widespread active substances used for various fields of use and employed as odor absorbers are activated carbon, silica gel, kieselguhr, Fuller's earth, zeolites, bentonites, minerals such as clays and montmorillonite. Further fixing and degrading substances are cyclodextrins, oxidizing agents, metal catalysts (e.g., TiO₂), enzymes, microorganisms and biocidal/biostatic compounds such as, for example, quaternary compounds (e.g., biocides such as benzalkonium saccharinate, cocoamidoamphopropionate, alkyl aminocarboxylate, and also antistatics, such as cetylmorpholinium ethosulfate).

In addition, deodorizing substances from one or more metal salts of an unbranched or branched, saturated or unsaturated, mono- or polyhydroxylated fatty acid having at least 16 carbon atoms and/or a resin acid with the exception of the alkali metal salts, and any mixtures of these salts with themselves, in particular, zinc salts in combinations with zinc salts of abietic acid or with zinc salts of other saturated or unsaturated hydroxylated fatty acids having 16 and more carbon atoms, and other active ingredients listed above, and in particular zinc salts of fatty acids, preferably of ricinoleic acid, alone or in combination with other active ingredients are known odor absorbers. Such a preparation is disclosed, for example, in DE B 17 92 074.

Zinc ricinoleate can chemically bind odor-intense organic substances with sulfur- or nitrogen-containing functional groups, such as, for example, mercaptans, thioethers, low molecular weight carboxylic acids, such as isovaleric acid, and also amines.

The ability of zinc ricinoleate to firmly chemically bind substances of this type therefore allows its use in industrial and private areas of application for reducing unpleasant household and industrial odors.

Zinc ricinoleate is a wax-like substance which has to be activated with water in order to be in a position to bind odors. However, since it is largely insoluble in water, it therefore has to be used in combination with solvents and solubility promoters in order to obtain effective preparations. The solvents used are in most cases mono- or polyhydric alcohols, optionally with the addition of water. Highly ethoxylated solubility promoters usually used are not able, even in high concentrations, to keep the zinc ricinoleate in solution by themselves, and consequently no flowable products have been obtained.

The solubility promoters described in the prior art are partial esters of di- or polyhydroxyalkanes, mono- and disaccharides, polyethylene glycols or alkanolamines with the ene adducts of maleic anhydride onto at least monounsaturated carboxylic acids having a chain length from 10 to 25 carbon atoms and acid numbers from 10 to 140, which are preferably buffered to a pH around 6.5 with amino and/or amido compounds, such as triethanolamine or glycol esters of aspartic acid and of glutamic acid through the formation of salt-like bonds.

Preparations containing these solubility promoters, however, are not flowable and the deodorant solutions formulated therefrom have a tendency, even at very low water contents, to cloud and precipitate out individual components. See, for example, DE-A-40 14 055, page 2, lines 50-52.

The efforts in the prior art therefore concentrate on providing improved solutions of zinc ricinoleate. The formulations prepared nowadays are a complex mixture of diverse constituents in which the actual active substance is only present in low concentrations.

Attempts to date to bind an odor absorber to substrates have led to a slight reduction in odor, although all of the prior art attempts did not exhibit the desired effectiveness. Since zinc ricinoleate is present in unactivated or complexed form on the substrates, only a small fraction of the odor-absorbing effect is usable.

The only commercial odor absorber products, which the applicants are aware of, are aqueous systems which ensure a significantly better odor eradication via specific activations. This can be deduced from the following publications:
Müller F., J. Peggau, H. Kuhn, Investigations on Zinc Ricinoleate as odour absorber with molecular dynamics calculations, Jorn. Com. Esp. Deterg. 30 (2000) 83-91.
Kuhn H., F. Müller, J. Peggau, R. Zekorn, Mechanism of the Odor-Absorption Effect of Zinc Ricinoleate. A Molecular Dynamics Computer Simulation, J. Surf. Deterg. 3 (2000) 335-343.
Müiller F., J. Peggau, T. Böhmer, New results on odor absorbtion with Zinc Ricinoleate, 41. wfk International Detergency Conference, Proceedings (2003) 130-137.

In many applications, however, aqueous systems cannot be used, such as, for example, in areas with very low or very high temperatures. In addition, aqueous systems always lead to waste water problems; for example, in highly diluted systems, extensive growth of microorganisms is observed. Some substrates, such as, for example, sheet silicates, exhibit complexing behavior, meaning that the zinc from the zinc ricinoleate remains firmly bonded to the substrate and is no longer available for a reaction with odorous and/or pollutant substances.

Similarly problematic are the binding ratios between zinc ricinoleate and unstructured, i.e., non-nanostructured, substrates which are employed to firmly bond zinc ricinoleate. Under some circumstances, the firmly bonded zinc ricinoleate would be inactive for the intended use purpose.

In view of the aforementioned problems with prior art odor absorbers, there is a continued need for new and improved odor absorbers which have the capacity to rapidly and efficiently remove odors.

### SUMMARY OF THE INVENTION

One object of the present invention is to provide a simple odor absorber, which is able to rapidly and permanently remove both existing and newly formed odors.

This object is achieved in the present invention by nanostructured surfaces as carriers and, incorporated therein, active zinc ricinoleate as an odor absorber.

The instant invention therefore provides solid odor absorbers including a solid carrier with a nanostructured surface and, incorporated therein, active zinc ricinoleate as an odor absorber.

The present invention therefore further provides solid odor absorbers wherein the solid carrier materials used are nanoporous materials which are able to fix water to the surface.

The present invention therefore also provides solid odor absorbers wherein the solid carrier materials used are nonporous metal oxides.

The present invention therefore even further provides solid odor absorbers wherein the solid carrier materials used are nanoporous anodically oxidized metals.

The instant invention still further provides solid odor absorbers wherein the solid carrier materials used are nanoporous anodically oxidized metals selected from the group of Cr, Hf, Nb, V, Ta and Zr.

The instant invention yet still further provides solid odor absorbers wherein the solid carrier materials used are nanoporous anodically oxidized aluminum and nanostructured titanium dioxide.

The instant invention also provides solid odor absorbers wherein the active substance used is at least one compound selected from the group of reactive odor absorbers, in particular, one or more metal salts of an unbranched or branched, unsaturated or saturated, mono- or polyhydroxylated fatty acid having at least 16 carbon atoms and/or a resin acid with the exception of the alkali metal salts, and from any mixtures of these salts with themselves or further active ingredients.

The invention provides even further solid odor absorbers wherein zinc salts are used in combinations with zinc salts of abietic acid or with zinc salts of other saturated or unsaturated hydroxylated fatty acids having 16 or more carbon atoms, and other active ingredients listed above, and, in particular, zinc salts of fatty acids, preferably the zinc salt of ricinoleic acid.

The invention additionally provides solid odor absorbers wherein the reactive odor absorber is combined with known solubilizers and/or activators, in particular, one or more anionic surfactants, amino acids in particular arginine and lysine, with the active ingredient and/or with further deodorizing active ingredients such as quaternary ammonium compounds or cyclodextrins.

The invention provides also for the use of the solid odor absorbers for deodorizing odorous or pollutant gases.

In the case of nanostructured titanium dioxide and nanostructured aluminum oxide whose oxide layer is prepared by anodic oxidation, a firm binding or incorporation of zinc ricinoleate does take place, but surprisingly without it being inactivated as a result and without additional water, solubility promoters and the other auxiliaries and additives required in the prior art.

Besides the classic applications, the depot effect resulting therefrom of these substrates also allows the use of zinc ricinoleate in areas where the use of aqueous systems is not possible due to high or low temperatures.

As a result of the depot effect, odorous and pollutant substances are firmly bonded to the substrate and can be disposed of in a targeted manner. Waste water problems and the growth of microorganisms can consequently be suppressed. As a result of the activation of the zinc ricinoleate on the abovementioned substrates and the thus higher odor-absorbing effect relative to unactivated zinc ricinoleate, a commercial use of these solid odor absorbers is possible.

### DETAILED DESCRIPTION OF THE INVENTION

As stated above, the present invention relates to solid odor absorbers which include anodic oxide carrier layers with active substances stored therein. Anodic oxide layers and the processes for their preparation form part of the known prior art.

To prepare these oxide layers used according to the present invention, all metals which form firmly adhering porous layers of suitable layer thickness and porosity with the anodically formed oxides and which are inert under application conditions, both to the gases to be absorbed and also to the environmental conditions are suitable. J. Electrochem. Soc. Jun. 1957, Vol. 104, No. 6, pages 339-346 describes, for example, the preparation of anodic oxide layers of Cr, Hf, Nb, V, Ta, Zr, Ti and Al.

Suitable carriers for the oxide layers are either the metals themselves or composites with any other carrier materials. Processes for producing these composites form part of the known prior art.

According to the instant invention, preference is given to nanoporous metallic materials such as aluminum/aluminum oxide (with a pore density of 10¹⁰-10¹² cm⁻² and a layer thickness of up to 250 μm) or titanium dioxide, such as, for example, E3® from Sachtleben GmbH, Duisburg.

The formation of the nanoporous aluminum oxide layer takes place under direct or alternating current in electrolytes whose pH is not 7, such as, for example, sulfuric acid, oxalic acid, phosphoric acid, boric acid, malonic acid and chromic acid. The aluminum to be anodized is surrounded by the electrolyte in question and connected as an anode of the cell. The cathode of the cell used is a metal which is inert to the electrolyte used in each case. By varying the voltage and temperature, and also through the choice of electrolyte, it is possible to control the pore size (diameter of the pore), the pore density (number of pores per square centimeter) and the hardness (fracture resistance) of the resulting oxide layer. Anodizing proceeds until the flow of current is interrupted or the oxide layer has grown to the maximum achievable thickness. Depending on the temperature prevailing during the anodizing, at temperatures from 0° C. to 5° C., a hard oxide layer is formed, and at temperatures above 5° C., a soft and flexible oxide layer is formed.

In this regard, reference is made to the publications by Parkhutik and Shershulski, J. Phys./D, 1992, Vol. 25, page 1258-1263; D. Höonicke, ALUMINIUM, 1989, Vol. 65, 11; reference is made to the entire contents.

Incorporation of the active substance in the porous oxide layer formed, consisting of hexagonal pores, can take place either with a melt or an aqueous solution of the active substance. The active substance is incorporated by treating the anodized aluminum sheets with 3% strength solution of zinc ricinoleate with solubilizers or in the melt of the active substance at 90° C. By increasing the treatment time, it is possible to increase the amount of incorporated active substance. The percentage amount of incorporated active substance is generally about 5 to 7% after 24 hours. Depending on the anodizing parameters, the mass fraction of the incorporated active substance can deviate up or down. Following subsequent drying, the active substance is firmly bonded to the nanostructured porous aluminum oxide surface.

The incorporation of the active substance into nanostructured titanium dioxide moldings can take place either with a melt or an aqueous solution of the active substance. The active substance is incorporated by treating the nanostructured titanium dioxide moldings in 3% strength solution of zinc ricinoleate with solubilizers or in the melt of the active substance at 90° C. By increasing the treatment time, it is possible to increase the amount of incorporated active substance. The percentage amount of the incorporated active substance is generally about 3 to 4% after 24 hours. Following subsequent drying, the active substance is firmly bonded to the nanostructured porous titanium dioxide moldings.

In addition, and besides the reactive odor absorbers, in particular, zinc ricinoleate, known solubilizers and/or activators, in particular, one or more anionic surfactants, in particular, sulfosuccinates, sulfosuccinamates and/or sulfosuccinamides, amino acids, in particular, arginine and lysine, and/or further deodorizing active ingredients, such as, for example, quaternary ammonium compounds or cyclodextrins, can be co-used.

The solid odor absorbers according to the present invention can be used for deodorizing odorous or pollutant gases, as can arise in air conditioning units, recirculated air units, household surfaces, waste containers, recycling containers, household appliances, cat litter, pets, pet sleeping areas, curtains, drapes, functional textiles, car interior upholstery, in public areas with a high density of people, such as, for example, in waiting rooms, bars, airports, hospitals. Here, the aluminum surfaces modified according to the invention can reduce, or decontaminate the bad odors without releasing liquid or aerosols into the air.

The following examples are provided to illustrate the odor absorbers of the present invention and to demonstrate some advantages thereof.

### EXAMPLE 1

Materials Used:
Solubilized zinc ricinoleate (Tego®Sorb A 30 from Goldschmidt GmbH).
Anodically oxidized aluminum, prepared by the anodic oxidation of 99.99% pure aluminum in 10% sulfuric acid over a period of 24 hours at a temperature of 10° C. and a voltage of 20 V. The porous oxide layers used had a pore density from 10¹⁰ to 10¹² cm⁻², a pore size from 10 to 250 nm and a layer thickness of up to 250 μm.
Sodium sulfide

One milliliter of a saturated sodium sulfide solution diluted 1:1 with distilled water (adjusted to pH 9) was initially introduced into a 100 ml single-neck round-bottomed flask with a wash bottle attachment, and one of the dry anodized Al sheets treated with Tego®Sorb to be investigated was immediately added. The anodized sheets were coated by incorporating the active substance in a 10% Tego®Sorb A 30 solution. In order to avoid direct contact between the sheet and sodium sulfide solution and, only to measure interaction between atmosphere and anodized surface, pieces of plastic were placed under the sheet as spacers.

Subsequently, and after certain times, the atmosphere in the flask was drawn off using a Dräager-Multiwarn II instrument, and the content of hydrogen sulfide was determined.

The hydrogen sulfide content of the atmosphere was determined for sheets coated with Tego®Sorb A 30, and also only anodized sheets and also untreated aluminum.

The first hydrogen sulfide measurement in the flask atmosphere was taken after ten minutes so that the formation of a homogeneous atmosphere was possible.

After just ten minutes, hydrogen sulfide could no longer be ascertained by the instrument in the atmosphere of the sheet coated with Tego®Sorb. In the other flasks with the uncoated or non-treated sections of aluminum, a hydrogen sulfide content of at least 30 ppm was still established after 24 hours.

The results obtained are tabulated below in summary.

TABLE 1
Comparison of anodized and coated and only anodized aluminum sheet

| | Content of H₂S [ppm] | |
|---|---|---|
| | anodized Al | Content of H₂S [ppm] |
| Time t [min] | with Tego ® Sorb A 30 | anodized, untreated Al sheet |
| 0 | — | — |
| 10 | 0 | 15 |
| 50 | 0 | 22 |
| 100 | 0 | 24 |
| 120 | 0 | 14 |

TABLE 2
Comparison of anodized and coated and only anodized aluminum sheet

| | | Content of H₂S [ppm] | |
|---|---|---|---|
| | | anodized Al with Tego ® | Content of H₂S [ppm] |
| Time t [min] | | Sorb A 30 | anodized, untreated Al sheet |
| 0 | | — | — |
| 15 | | 0 | 24 |
| 45 | | 0 | 26 |
| 120 | | 0 | 33 |
| 24 | h | 0 | >100 |

TABLE 3
Comparison of anodized noncoated and untreated aluminum sheet

| | | Content of H₂S [ppm] | Content of H₂S [ppm] |
|---|---|---|---|
| Time t [min] | | Anodized, untreated Al sheet | Al sheet untreated |
| 0 | | — | — |
| 10 | | 31 | 24 |
| 40 | | 31 | 32 |
| 70 | | 29 | 33 |
| 120 | | 30 | 36 |
| 24 | h | 27 | 29 |

The sheets compared in Tables 1, 2 and 3 were anodized under identical conditions, at 10° C. and 20 V, in 10% sulfuric acid.

Results:

The results in Tables 1 to 3 clearly show that effective removal of hydrogen sulfide is found only with the anodized aluminum sheets in combination with incorporated Tego®Sorb. A reduction or removal of the odor and pollutant hydrogen sulfide results neither when using untreated aluminum, nor when using anodized aluminum. The effectiveness of Tego®Sorb in anodically prepared porous aluminum oxide layers was thus demonstrated.

### EXAMPLE 2

Materials Used:
Solubilized zinc ricinoleate (Tego®Sorb A 30 from Goldschmidt GmbH).
Anodically oxidized aluminum prepared by the anodic oxidation of 99.99% pure aluminum in 10% sulfuric acid over a period of 24 hours at a temperature of 10° C. and a voltage of 20 V. The porous oxide layers used had a pore density from 10¹⁰ to 10¹² cm⁻², a pore size from 10 to 250 nm and a layer thickness of up to 250 μm.
Ammonia 25% strength.

One milliliter of a 1:20 or 1:10 dilution of 25% ammonium hydroxide was placed in each case with an anodized aluminum sheet coated with Tego®Sorb A 30 into a screw-lid vessel. Two further screw-lid vessels only with the ammonia dilutions and without aluminum sheet served as a blank sample. In order to avoid direct contact between the sheet and ammonia, and only to measure interaction between atmosphere and anodized surface, pieces of plastic were placed under the sheet as spacers.

After a period of 24 h, in the round-bottomed flasks, aluminum sheets coated with Tego®Sorb A 30, a reduction in the pH from pH 11 to pH 10 was evident, whereas in the other flasks the pH was still 11.

The resulting pH of the aqueous ammonia phase was determined using universal indicator strips from Merck (pH 0 to 14).

### EXAMPLE 3

Materials Used:
Solubilized zinc ricinoleate (commercial product Tego®Sorb 80 from Goldschmidt GmbH).
Titanium dioxide (Titanium dioxide E3 from Sachtleben Chemie GmbH)
Ammonia 25% strength.

In each case, 4 g of titanium dioxide coated with Tego®Sorb 80 and 1 ml of a 1:10 and 1:20 dilute 25% strength ammonia solution were initially introduced into a screw-lid vessel. Two further screw-lid vessels only with the ammonia dilutions and without titanium dioxide served as a blank sample.

So that no points of contact between dry titanium dioxide and liquid resulted, the titanium dioxide was separated by a small container within the screw-lid vessel.

After 18 h, the pH in the four vessels was measured. The two screw-lid vessels without titanium dioxide showed a pH of 10 and a pungent ammonia odor was still detected.

The two screw-lid vessels with titanium dioxide showed a reduction in the pH only via the gas phase. The resulting pH in the case of the 1:10 dilution after 18 h was pH 8 to 9, and in the case of the 1:20 dilution, pH 7 to 8.

In order to also make the results visible, pH universal indicator paper strips were hung into each of the screw-lid vessels. The resulting pH was determined using universal indicator strips from Merck (pH 0 to 14).

Results:

The results from example 2 and 3 clearly show a reduction in the pH of the gas phase and in particular of the liquid in the screw-lid vessels or single-neck round-bottomed flasks. The results clearly show that titanium dioxide moldings or anodized aluminum sheets coated with Tego®Sorb lead to a reduction in ammonia via the gas phase.

### EXAMPLE 4

Materials Used:
Solubilized zinc ricinoleate (Tego®Sorb A 30 from Goldschmidt GmbH)
Titanium dioxide (Titanium dioxide E3 from Sachtleben Chemie GmbH)
Sodium sulfide.

One milliliter of a saturated sodium sulfide solution diluted 1:1 with distilled water (adjusted to pH 9) was initially introduced into a 100 ml single-neck round-bottomed flask with a wash bottle attachment, and the titanium dioxide moldings coated with Tego®Sorb A 30 to be investigated were immediately added. The titanium dioxide moldings were coated by incorporating the active substance in a 10% Tego®Sorb A 30 solution. In order to avoid direct contact between the sheet and sodium sulfide solution and, to measure only interaction between atmosphere and anodized surface, pieces of plastic were placed under the titanium dioxide as spacers.

Then, after certain times, the atmosphere in the flask was drawn off by a Dräger-Multiwam II instrument and the content of hydrogen sulfide was determined.

The hydrogen sulfide content of the atmosphere was determined for titanium dioxide coated with Tego®Sorb A 30, and also uncoated titanium dioxide and only sodium sulfide solution as blank sample.

The first hydrogen sulfide measurement in the flask atmosphere was made after five minutes so that the formation of a homogeneous atmosphere was possible.

After just five minutes, hydrogen sulfide was no longer ascertained by the instrument in the atmosphere of the titanium dioxide molding coated with Tego®Sorb A 30. In the other flasks with the uncoated titanium dioxide moldings or the blank sample, hydrogen sulfide was still established even after 24 hours.

The results obtained are tabulated below in summary.

TABLES 4
Comparison of coated/noncoated titanium dioxide (Sachtleben)

| | | Content of | Content of H₂S [ppm] | Content of H₂S |
|---|---|---|---|---|
| | | H₂S [ppm] | titanium dioxide | [ppm] blank sam |
| Time t | | titanium dioxide | coated with | (without |
| [min] | | untreated | Tego ® Sorb A 30 | titanium dioxide) |
| 0 | | — | — | — |
| 5 | | 14 | 0 | 24 |
| 30 | | 15 | 0 | 24 |
| 60 | | 12 | 0 | 15 |
| 120 | | 10 | 0 | 10 |
| 210 | | 8 | 0 | 6 |
| 270 | | 10 | 0 | 12 |
| 24 | h | 3 | 0 | 12 |

Results:

The results in Table 4 clearly show that effective removal of hydrogen sulfide was evident only in the case of the titanium dioxide moldings treated with Tego®Sorb. No reduction or removal of the odor and pollutant hydrogen sulfide resulted when using untreated titanium dioxide moldings.

While the present invention has been particularly shown and described with respect to preferred embodiments thereof, it will be understood by those skilled in the art that the foregoing and other changes in forms and details may be made without departing from the spirit and scope of the present invention. It is therefore intended that the present invention not be limited to the exact forms and details described and illustrated, but fall within the scope of the appended claims.

What is claimed is:

1. A solid odor absorber comprising a solid carrier having a nanostructured surface and, incorporated therein, active zinc ricinoleate as an odor absorber.

2. The solid odor absorber as claimed in claim 1, wherein the solid carrier comprises a nanoporous material which is able to fix water to a surface thereof.

3. The solid odor absorber as claimed in claim 1, wherein the solid carrier is a nanoporous metal oxide.

4. The solid odor absorber as claimed in claim 1, wherein the solid carrier is a nanoporous anodically oxidized metal.

5. The solid odor absorber as claimed in claim 1, wherein the solid carrier is a nanoporous anodically oxidized metal selected from the group consisting of Cr, Hf, Nb, V, Ta and Zr.

6. The solid odor absorber as claimed in claim 1, wherein the solid carrier is nanoporous anodically oxidized aluminum or nanostructured titanium dioxide.

7. The solid odor absorber as claimed in claim 1, further comprising at least one active substance selected from the group consisting of one or more metal salts of an unbranched or branched, unsaturated or unsaturated, mono- or polyhydroxylated fatty acid having at least 16 carbon atoms and/or a resin acid with the exception of alkali metal salts, and any mixtures of these salts with themselves or further active ingredients.

8. The solid odor absorber as claimed in claim 7, wherein said metal salts are zinc salts that are used in combinations with zinc salts of abietic acid or with zinc salts of other saturated or unsaturated hydroxylated fatty acids having 16 or more carbon atoms.

9. The solid order absorber as claimed in claim 8, wherein said zinc salts are zinc salts of ricinoleic acid

10. The solid odor absorber as claimed in claim 1, wherein the odor absorber is combined with a solubilizer, an activator or combinations thereof.

11. The solid odor absorber as claimed in claim 1, wherein said odor absorber is combined with a least one further deodorizing active ingredient.

12. The solid odor absorber as claimed in claim 11, wherein said at least one further deodorizing active ingredient is a quaternary ammonium compound or cyclodextrin.

13. A method for deodorizing odorous or pollutant gases comprising contacting a deodorizing odorous or pollutant gas with a solid odor absorber, said solid odor comprising a solid carrier having a nanostructured surface and, incorporated therein, active zinc ricinoleate.
